(19) United States
(12) Patent Application Publication
(10) Pub. No.: US 2014/0058388 A1
(43) Pub. Date: Feb. 27, 2014

(54) SURGICAL SYSTEM FOR BONDING BODY TISSUE AND CONTROL METHOD FOR SAID SURGICAL SYSTEM

(71) Applicants: Dieter Weisshaupt, Immendingen (DE); Christoph Rothweiler, Donaueschingen (DE); Patrick Heizmann, Huefingen (DE)

(72) Inventors: Dieter Weisshaupt, Immendingen (DE); Christoph Rothweiler, Donaueschingen (DE); Patrick Heizmann, Huefingen (DE)

(73) Assignee: Aesculap AG, Tuttlingen (DE)

(21) Appl. No.: 14/001,950

(22) PCT Filed: Mar. 8, 2012
(86) PCT No.: PCT/EP2012/054047

§ 371 Date: Nov. 5, 2013

(30) Foreign Application Priority Data
Mar. 17, 2011 (DE) 10 2011 001 372.5

(51) Int. Cl. A61B 18/14

(52) U.S. Cl. CPC A61B 18/1445
USPC 606/52

(57) ABSTRACT

A surgical system for bonding body tissue includes a surgical instrument having tool elements movable relative to each other, each of which includes an HF electrode defining a minimum distance from each other, being opposed to each other and facing each other in an approximating position of the tool elements so as to bond body tissue held between the tool elements, and including a cutting element for transecting and/or resecting body tissue, the cutting element being adapted to be brought from a tissue bonding position, in which body tissue can be bonded to the tool elements and in which the cutting element cannot contact body tissue, into a cutting position such that faulty operation of the system is minimized. The instrument includes a means for retaining the cutting element in the tissue bonding position. An improved control method for a surgical system for bonding body tissue is also described.

### RELATED APPLICATIONS

This application is the U.S. national phase of International Application No. PCT/EP2012/054047, filed Mar. 8, 2012, which claims the benefit of priority of German Application No. 10 2011 001 372.5, filed Mar. 17, 2011, the contents of which are incorporated by reference herein in their entirety and for all purposes.

### FIELD

The present invention relates to a surgical system for bonding body tissue comprising a surgical instrument having at least two tool elements movable relative to each other, each comprising at least one HF electrode defining in an approximated position of the tool elements a minimum distance from each other, being opposed to each other and facing each other so as to bond body tissue held between the tool elements due to HF current being applied, and comprising a cutting element for transecting and/or resecting body tissue, the cutting element being adapted to be brought from a tissue bonding position, in which body tissue can be bonded to the tool elements and in which the cutting element cannot contact body tissue, into a cutting position.

Furthermore the present invention relates to a control method for a surgical system for bonding body tissue, the system comprising a surgical instrument having at least two tool elements movable relative to each other, each of which comprises at least one HF electrode which in an approximated position of the tool elements define a minimum distance from each other, are opposed to each other and are facing each other so as to bond body tissue held between the tool elements due to HF current being applied, and comprising a cutting element for transecting and/or resecting body tissue, which cutting element is adapted to be brought from a tissue bonding position in which body tissue can be bonded to the tool elements and in which the cutting element cannot contact body tissue into a cutting position.

### BACKGROUND

Surgical systems and control methods of the type described in the beginning are known, for example, from DE 20 2010 013 150 UI. They are used especially in surgical interventions in which so called “side-to-side”, “end-to-end” or “end-to-side” anastomoses are performed. For bonding the body tissue HF current of defined intensity is applied to the parts to be bonded to each other. The bonding made in this way is also referred to as “welding” or “sealing”.

After bonding the tissue excessive tissue can be resected by means of the cutting element or the now bonded tissue parts can be partly transected again so as to open especially a newly formed cavity. What is important is that the cutting element is activated only when the bonding of the body tissue is completed. Otherwise, an undesired damage of the patient in the area of the operation site may occur.

Therefore, it is an object of the present invention to improve a surgical system as well as a control method of the type described in the beginning so that a faulty operation of the system is most probably excluded.

### SUMMARY

According to embodiments of the invention, this object is achieved in a surgical system of the type described in the beginning in that the instrument comprises a retaining means for retaining the cutting element in the tissue bonding position.

The retaining means in particular permits preventing the use of the cutting element as long as transecting or resecting body tissue is not or not yet desired. Thus operating surgeons who have performed “side-to-side”, “end-to-end” or “end-to-side” anastomoses by conventional clip suture instruments so far can be offered a surgical system for bonding body tissue using HF current with the same handling. Operating surgeons using clip suture instruments are accustomed to seizing the tissue first with the clip suture instrument, then to bonding the tissue parts to be bonded by clips and after clip bonding of the body tissue automatically transecting the latter mechanically by a cutting element of the clip suture instrument. The design structure of conventional clip suture instruments ensures that the cutting element can be activated only when at least one clip has been set. The retaining means suggested according to the invention especially can ensure that now also the handling of a surgical system described in the beginning can correspond to the handling of a clip suture instrument which is important especially because the instruments for bonding body tissue by HF current externally differ only little from conventional clip suture instruments and therefore an operating surgeon intuitively uses a surgical instrument operated with HF current just as a clip suture instrument.

The structure of the surgical system becomes especially simple when the retaining means comprises at least one retaining element which in the tissue bonding position blocks or prevents activation of the cutting element. The at least one retaining element can especially guarantee in a simple manner that the cutting element is not moved in an undesired way or HF current is applied thereto for cutting the body tissue without this being actually desired.

In an especially simple manner the cutting element can be retained in the tissue bonding position when the at least one retaining element is engaged at least partly with the cutting element in the tissue bonding position in a force-fit and/or form-fit manner. In other words, the retaining means thus is adapted to retain the cutting element especially mechanically in the tissue bonding position.

An especially simple construction of the system can be achieved in particular by the fact that the cutting element comprises first and second retaining elements, that the cutting element comprises or supports the first retaining element and that the instrument comprises or supports the second retaining element. Both retaining elements can thus be provided at the instrument, the first retaining element being preferably arranged or formed at the cutting element.

It can moreover be of advantage when the first and second retaining elements are movable relative to each other from a retaining position, in which they are engaged in force-fit and/or form-fit, into a release position in which they are disengaged. This configuration permits, by simple movement of the retaining elements relative to each other, either retaining the cutting element in the tissue bonding position or releasing the cutting element so as to resect or transect body tissue.

It is especially advantageous when the first and second retaining elements are movable relative to each other in a direction extending transversely, preferably normal to a direction of motion of the cutting element upon transition from the tissue bonding position to a tissue cutting position in which the cutting element protrudes at least in portions from at least one of the at least two tool elements so as to transect body tissue. An especially safe locking of the cutting element in the tissue bonding position can be achieved in a simple way by the directions of motion of the retaining elements oriented transversely relative to each other as well as of the cutting element.

The configuration of the retaining means becomes especially simple when either of the two retaining elements is designed in the form of a projection and the other retaining element takes the form of a recess. In particular the two retaining elements can be formed to be corresponding to each other so that a force-fit and/or form-fit interaction of the retaining elements in the tissue bonding position is made possible. Basically both parts can be movably arranged at the instrument, for example the projection or else a part of the instrument carrying the recess.

In an especially simple manner the retaining means can be operated when either of the two retaining elements is coupled to a drive arranged at the instrument for moving the retaining element. Thus, by the drive at least one of the retaining elements can be moved so as to move the latter relative to each other so that the retaining means can be transferred from the retaining position into the release position.

It is favorable when the drive is an electromotive, pneumatic, hydraulic, magnetic, electromagnetic or piezoelectric drive. Those drives are adapted to easily and safely move a retaining element at the instrument in defined manner.

The drive preferably comprises a magnet, a solenoid, a relay, a step motor, a piezo element, an electromechanical actor or a servomotor. Drives equipped in this way especially enable the retaining means not only to be operated mechanically but also to be controlled electrically or electronically.

In a simple way body tissue can be transected, when the cutting element is a mechanical cutting element or a HF cutting element. On the one hand, a mechanical cutting element in the form of a blade can be used for transecting body tissue. On the other hand, it is also possible to transect body tissue by means of current. In this case a movement of the cutting element relative to the tool elements is not absolutely necessary.

Advantageously the instrument comprises an operating means for moving the tool elements relative to each other. This enables an operating surgeon in a simple manner to seize body tissue to be bonded and to bond the same by applying current. Furthermore, the operating means can also comprise an operating element for moving the cutting element so that an operating surgeon can manually move the cutting element for example from a position withdrawn vis-à-vis the tool elements into a position advanced vis-à-vis the same so that the cutting element can contact the body tissue to be transected. However, this movement of the cutting element can only take place when the retaining means does not retain the cutting element in the tissue bonding position.

The handling of the instrument becomes especially simple when the operating means is arranged or formed at a proximal end thereof.

It is basically imaginable to arrange the cutting element to be immobile relative to at least one of the tool elements or else to both of them. Preferably the cutting element is arranged to be movable relative to either of the tool elements, however. This permits transferring the cutting element especially from a position withdrawn vis-à-vis the at least one tool element into a position projecting vis-à-vis said tool element in order to allow body tissue being transected mechanically by the cutting element which may be in the form of a cutting knife having a cutting edge, for example.

It is beneficial for the surgical system to comprise at least one HF current generator adapted to be optionally connected to the HF electrodes and/or the cutting element in an electrically conducting manner. Thus the HF current generator is adapted to apply HF current to body tissue, namely on the one hand for bonding the body tissue and optionally also for transecting the same.

It is of advantage for the surgical system to comprise at least one process control means for controlling the HF current generator and/or the instrument. By the process control means it is especially possible to control the HF current generator and the instrument, respectively, so that the body tissue can be sealed and welded, respectively, in an optimum and defined manner. In particular, it can be considered that for preventing failure of the sealing or the welding the parameters acting on the body tissue are detected and controlled.

For controlling the HF current generator it is especially beneficial when the at least one process control means comprises a switching means for switching at least one HF output of the at least one HF current generator. In this way, the at least one process control means is adapted to directly influence a function, especially current supply, of the HF generator.

An especially compact design of the surgical system can be obtained in particular by the HF current generator comprising the at least one process control means. For example, the process control means as well as the HF current generator can be arranged in a common housing.

It is advantageous for the at least one process control means to be configured to control the retaining means. This permits a defined interaction of the process control means and the retaining means. Thus it can be achieved that e.g. a release of the cutting element permitting a movement of the same to transect body tissue is possible only when a successful bonding of the body tissue controlled by the process control means has been performed by means of HF current.

It is favorable when the process control means controls the HF current generator connected to the instrument during a tissue bonding cycle so that body tissue can be bonded between the electrodes in a defined manner. Such configuration of the process control means can ensure that no failure of the tissue bonding will occur. In particular, upon bonding the body tissue one or more parameters acting on the same can be detected and controlled.

It is of advantage when sensors for determining process parameters are arranged on the instrument and when the process control means treats the process parameters detected by the sensors upon running the tissue bonding cycle. For example, the sensors are adapted to determine a temperature as well as a resistance of the tissue during the tissue bonding cycle and, in response to the values thereof, current application by the HF current generator can be appropriately controlled so as to obtain a defined sealing or welding of the body tissue and to avoid destruction of the same.

Of preference, the temperature and/or the tissue resistance are detected as process parameters. In particular, by measuring said process parameters it can be evaluated very reliably, as a rule, whether the body tissue has been bonded already and, if so, in a desired way.

According to a preferred embodiment of the invention, it can be provided that the process control means controls the retaining means so that the cutting element is released only after completion of a tissue bonding cycle so as to be able to bring, especially move it from the tissue bonding position into the tissue cutting position. Such configuration of the process control means permits to ensure that only after successful bonding of the body tissue the cutting element can be used at all and a transecting or resecting can be performed by the same, where appropriate.

It is of advantage when the process control means is configured so that intensity and/or duration of current feed for the HF electrodes can be adjusted. By such process control means for example desired process parameters can be adjusted in advance and can be set as desired values.

It is beneficial for the process control means to comprise a temperature measuring means for measuring a HF electrode temperature and/or a tissue temperature.

Measuring the HF electrode temperature and the tissue temperature, respectively, as process parameters in particular permits to detect and evaluate the progress as well as the quality of the tissue bonding already during the tissue bonding cycle.

Furthermore, it can be advantageous for the process control means to comprise a resistance measuring means for measuring a tissue resistance. Measuring the tissue resistance as a process parameter also has the advantage that the progress as well as the quality of the tissue bonding can thus be detected and evaluated in a simple manner.

In accordance with the invention, the object set forth in the beginning is furthermore achieved with a control method of the type as described in the beginning by the fact that the cutting element is retained in the tissue bonding position until a tissue bonding cycle has been carried out in which body tissue has been bonded between the electrodes in a defined manner.

The suggested control method, which is especially adapted to be employed for controlling one of the afore-described surgical systems, allows in a simple way to ensure that the cutting element can only be activated, especially moved, and used for transecting body tissue when a proper tissue bonding cycle preferably monitored by the surgical system has been carried out.

### BRIEF DESCRIPTION OF THE DRAWING FIGURES

The following description of preferred embodiments of the invention serves, in connection with the drawings, for more detailed illustration, showing in:

FIG. 1 a schematic representation of a surgical system according to the invention in a tissue bonding position;

FIG. 2 an enlarged cut-out view of the area A of FIG. 1; and

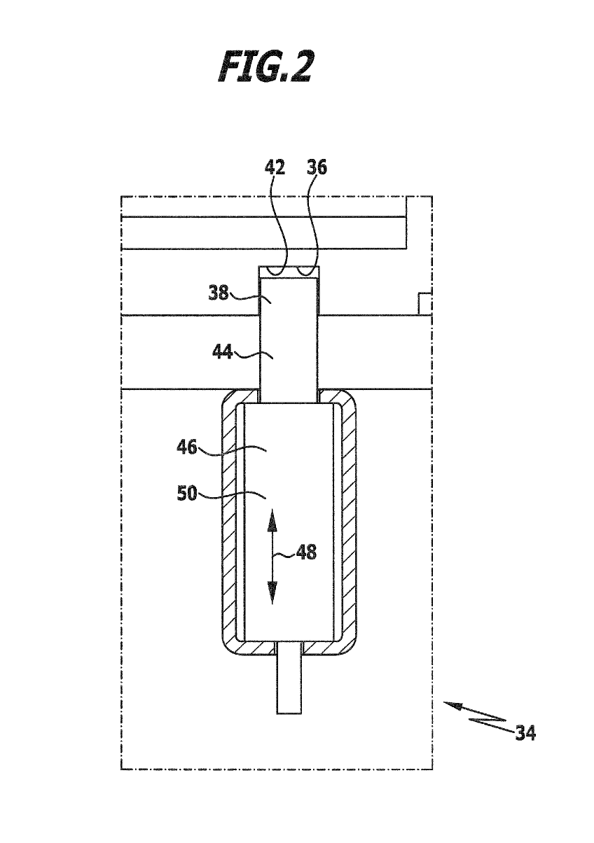

FIG. 3 a schematic view of the surgical instrument, which is shown in FIG. 1 in a tissue bonding position, in a tissue transecting position.

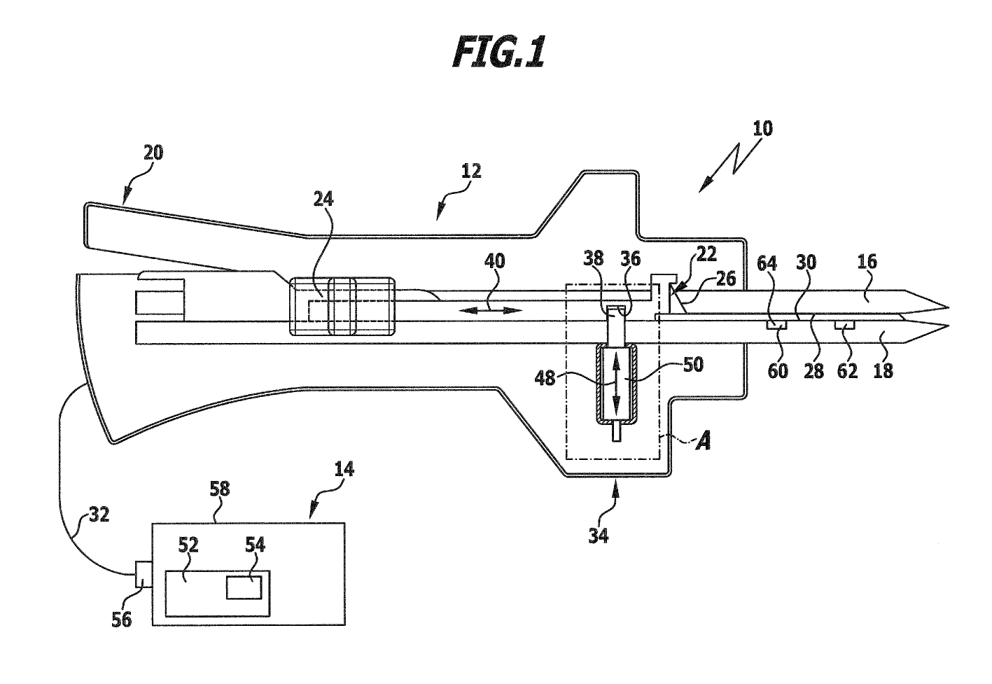

In FIG. 1 a surgical system according to the invention is schematically shown and is denoted with the reference numeral 10. It comprises a surgical instrument 12 and a HF current generator 14.

The instrument 12 comprises two preferably pivoting tool elements 16 and 18 movable relative to each other which can be moved by means of an operating means 20 constituting a proximal end of the instrument 12. Moreover the instrument 12 comprises a cutting element 22 in the form of a sliding scalpel. It is movably supported at the instrument 12 by means of a cutting element support in a way not shown in detail. For moving the cutting element 22 a sliding button 24 is provided in a form protruding from an outside of the instrument 12. For example, it can be shifted in the distal direction by the thumb of an operating surgeon, wherein the cutting element 22 having a cutting edge 26 facing in the distal direction can be simultaneously moved, namely relative to the tool elements 16 and 18 such that at least the cutting edge 26 protrudes from the same and body tissue can be transected in this way.

Each of the tool elements 16 and 18 comprises a HF electrode 28 and 30, respectively, defining a minimum distance, are opposed to each other and are facing each other in an approximating position of the tool elements 16 and 18. HF current supplied from the HF current generator 14 via a connecting line 32 to the instrument 12 can be applied to them.

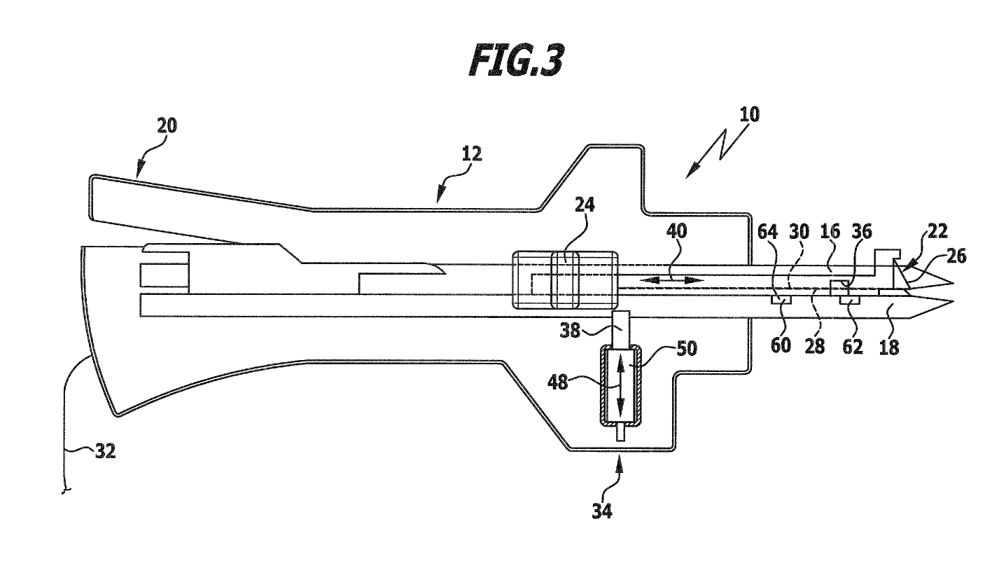

Furthermore, the instrument 12 comprises a retaining means 34 for retaining the cutting element 22 in a tissue bonding position in which body tissue can be connected to the tool elements 16, 18 and in which the cutting element 22 preferably is not adapted to contact body tissue. The retaining means 34 comprises first and second retaining elements 36 and 38. The first retaining element 36 is preferably associated with the cutting element 22 or is comprised by the same and is supported by the same, respectively. The instrument 12 further also comprises the second retaining element 38 or supports the latter. The retaining elements 36, 38 are configured to be movable relative to each other and can be brought from a retaining position as schematically shown in FIG. 1, in which they are engaged in force-fit and/or form-fit, into a release position as schematically shown in FIG. 3, in which they are disengaged. The first retaining element 36 is designed in the form of a recess 42 opened facing transversely to a displacing direction 40 of the cutting element 22. The second retaining element 38 is designed in the form of a pin-shaped end 44 being adapted to be inserted in the recess 42 in the retaining position and thus blocking a movement of the cutting element 22 in the displacing direction 40.

In order to be able to move the retaining elements 36, 38 relative to each other in a simple way, the second retaining element 38 is coupled to a drive 46. The latter enables the second retaining element 38 to move in a direction 48 extending transversely, preferably perpendicularly, to the displacing direction 40. The second retaining element 38 can thus be moved into the recess 42 as well as out of the same again in a simple manner.

The drive 46 is configured as a magnetic drive 50 and comprises a lifting magnet adapted to displace the pin-shaped second retaining element 38 in the direction 48. In a home position of the drive 46 the second retaining element 38 adopts the retaining position. Hence, if no current is fed to the lifting magnet, the retaining elements 36, 38 are engaged. For transferring the retaining means 34 from the tissue bonding position into the release position the drive 46 has to be activated, i.e. current has to be fed to the lifting magnet. The retaining means 34 is thus designed as an electromechanical stop. As further types of drive alternatively also electromotive, pneumatic, hydraulic or else piezoelectric drives can be provided instead of the magnetic drive 50.

As an alternative to the afore-described mechanical cutting element 22, also a HF cutting element can be provided. This means that for transecting body tissue HF current supplied by the HF current generator can be applied to the cutting element 22. In such case it is not absolutely necessary to arrange the cutting element 22 relative to the tool elements 16, 18 to be movable at the instrument 12. The sliding button 24 can especially exert the function of an electric switch or pushbutton and need not necessarily be mechanically coupled to the cutting element 22.

A process control means 52 serves for controlling the HF current generator 14. It is also adapted to control the use of the instrument 12. The process control means 52 further preferably includes a switching means 54 for switching at least one HF output 56 of the current generator 14. The process control means 52 can be arranged especially in a housing 58 of the HF current generator 14.

The process control means 52 is configured so that it can be used for controlling the retaining means 34. This is achieved in that the process control means 52 controls the retaining means 34 such that the cutting element 22 is released only after completion of a proper tissue bonding cycle. During a tissue bonding cycle body tissue can be bonded between the electrodes 28, 30 in a defined manner by applying HF current. This is controlled by the process control means 52, namely in particular taking into account sensors 60 and 62 arranged at the instrument 12 for determining process parameters. In this case especially a sensor 60 in the form of a temperature measuring means 64 for determining a temperature of the HF electrodes 28, 30 and/or a tissue temperature can be concerned. Furthermore the sensor 62 can be in the form of a resistance meter 66 for measuring a tissue resistance. The process parameters detected in the afore-described manner are preferably processed by the process control means 52 so as to appropriately control the tissue bonding cycle. It can be ensured by detecting the process parameters that the tissue is bonded in a defined way so as to be able to exclude later failure of the bonding, if possible. Moreover, in this way also current feed can be restricted, if required, so that destruction of the tissue to be bonded can be prevented.

In order to further avoid faulty operation of the system 10, the control of the retaining means 34 by the process control means 52 ensures that the cutting element 22 is released only after completion of a properly run tissue bonding cycle so as to be able to activate, for example to move the same from the tissue bonding position into the tissue transecting position, or to apply HF current for cutting to the same.

Individual functions of the system 10 can be monitored especially by means of safety inquiries by the process control means 52. A tissue bonding cycle can be controlled so that opening and closing, i.e. simply moving the tool elements 16 and 18 relative to each other, is always permitted. Furthermore, it can also be constantly allowed to transfer the tool elements 16 and 18 into the tissue bonding position, to bond the tissue and subsequently to re-open the tool elements 16 and 18 so as to release the tissue seized there between. Closing of the tool elements 16, 18, subsequent bonding of the body tissue being controlled and verified by the HF current generator 14 as well as afterwards cutting of the body tissue after releasing the cutting element 22 by the retaining element 34 is to be equally possible. However, what is not admissible is closing of the tool elements 16, 18 and a subsequent direct activation of the cutting element 22 without a successful HF coagulation controlled by the process control means 52. What is not to be possible, either, is the activation of the cutting element 22, when after closing the tool elements 16, 18 a bonding of the body tissue has taken place which has not been controlled by the process control means 52, though. Since coagulation with an apparatus not belonging to the system 10 cannot be verified by the system, the retaining means 34 remains deactivated in such manner that the cutting element 22 is not released and thus can neither be moved nor actuated.

The afore-described preferred embodiments of a surgical system 10 as well as a surgical control method permit a regulated operating process which enables an operating surgeon to safely produce tissue anastomoses. It can be achieved especially by the afore-described systems and methods that the tissue seized by the instrument 12 cannot be transected before the process control means 52 has verified the tissue bonding process as being successfully performed.

In order to be able to verify whether the instrument 10 is provided in the tissue bonding position or in the cutting position, optionally an inductive monitoring of the lifting magnet not shown in detail can be integrated, for example. As an alternative, also the use of a magnetic drive 50 in the form of a so called bi-stable lifting magnet is possible which each time keeps stable either of the two positions, i.e. either the tissue bonding position or the cutting position, and then adopts the respective other position due to current feed and by reversing the polarity.

Furthermore, a protection can be additionally provided which monitors a position of the operating unit 20 for example via an appropriate sensor, e.g. a pushbutton, so as to prevent that the sealing process can be carried out while the operating unit 20 is opened. The operating unit 20 can especially be configured so that it is coupled to an operating member for HF current operation, for example a HF pushbutton, in a logic AND inquiry. In this way it can be ensured that current feed to the HF electrodes 28 is only possible when the operating unit is closed and the HF pushbutton is simultaneously actively pressed.

1.-10. (canceled)

11. A surgical system for bonding or transecting body tissue by a surgical instrument having two instrument branches each equipped with at least one HF electrode which are supported on a branch holder, an instrument handle for opening and closing the instrument branches and a cutting element equally supported on the branch holder, preferably the instrument handle, separately from the instrument branches, wherein the surgical instrument is equipped with a retaining means in the form of a blocking mechanism of the cutting element which in the non-activated or non-operated condition mechanically completely locks a cutting movement of the cutting element and can be actively transferred into an unlocking condition to permit a cutting motion, and wherein active unlocking of the retaining means is only possible when a predefined tissue bonding cycle is completed, preferably when the two instrument branches are in the open condition and/or no current is fed to the HF electrodes at the instrument branches.

12. The surgical system according to claim 11, wherein the retaining means consists of a locking bar or stud which is movable at an angle, preferably normal to the direction of motion of the cutting element and can be engaged in an undercut preferably in the form of a recess at the cutting element for locking the motion of the cutting element.

13. The surgical system according to claim 12, wherein the locking bar or stud is operatively connected to a drive moving the locking bar or stud into its locked and unlocked positions.

14. The surgical system according to claim 13, wherein the drive has an electromagnetic, pneumatic, hydraulic, magnetic, electromotive or piezoelectric design.

15. The surgical system according to claim 11, wherein the cutting element is a mechanically acting scalpel or a HF knife.

16. The surgical system according to claim 13, further comprising a process control means for HF current feed to the HF electrodes and optionally to the cutting element.

17. The surgical system according to claim 13, wherein the process control means controls the drive so that unlocking of the retaining means is blocked when the branches are closed or current is fed to the HF electrodes.

18. The surgical system according to claim 16, wherein the process control means controls the retaining means such that the cutting element is released only after completion of a tissue bonding cycle so as to be able to bring, especially move, it from the tissue bonding position into the tissue transecting position.

19. The surgical system according to claim 16, characterized in that a tissue bonding cycle is deemed to be completed according to definition when a defined sealing or welding of the body tissue is obtained.

20. A control method for a surgical system for bonding or transecting body tissue comprising the steps of:
retaining a cutting element in its non-active tissue bonding position until the end of a tissue bonding cycle carried out by the branch-supported HF electrodes is displayed and
activating the drive for unlocking the retaining means upon receiving a signal indicative of the end of a tissue bonding cycle as well as independently of operating the cutting element.
